(19) United States
(12) Plant Patent Application Publication
(10) Pub. No.: US 2014/0310840 P1
(43) Pub. Date: Oct. 16, 2014

(54) Tulbaghia plant named 'Hinetul1'

(50) ;

(71) Applicant: HINES GROWERS, INC., Fallbrook, CA (US)

(72) Inventor: David Hernandez, Orange, CA (US)

(73) Assignee: Hines Growers, Inc., Fallbrook, CA (US)

(21) Appl. No.: 13/986,204

(22) Filed: Apr. 11, 2013

(51) Int. Cl. A01H 5/00

(52) U.S. Cl. PLT/263.1

(57) ABSTRACT

A Tulbaghia plant particularly distinguished by purple inflorescences and purple and white variegated foliage, is disclosed.

### GENUS AND SPECIES

Tulbaghia violacea

### VARIETY DENOMINATION

‘Hinetul1’

### BACKGROUND OF THE NEW PLANT

The present invention comprises a new and distinct variety of Tulbaghia, botanically known as Tulbaghia violacea, and hereinafter referred to by the variety name ‘Hinetul1’. The new variety originated as a mutation in an unnamed and unpatented bed of Tulbaghia violacea in June 2002 in a nursery in Irvine, Calif.

The new plant was first asexually propagated in Irvine, Calif. in April 2003 and has been asexually reproduced by divisions for approximately 9 years in Irvine, Calif. The present invention has been found to retain its distinctive characteristics through successive asexual propagations by divisions.

‘Hinetul1’ has not been made publicly available or sold more than one year prior to the filing of this application.

### SUMMARY

The following are the most outstanding and distinguishing characteristics of the new variety when grown under normal horticultural practices in Irvine, Calif.

1. Large, purple inflorescences; and

2. Purple and white variegated foliage.

### DESCRIPTION OF THE PHOTOGRAPHS

This new Tulbaghia plant is illustrated by the accompanying photographs which show the overall plant habit, foliage and inflorescence of the plant. The colors are as true as can be reasonably obtained by conventional photographic procedures. The photographs are of plants grown outdoors in Irvine, Calif. in 2012.

FIG. 1 shows a the whole plant, including the habit, foliage and inflorescence.

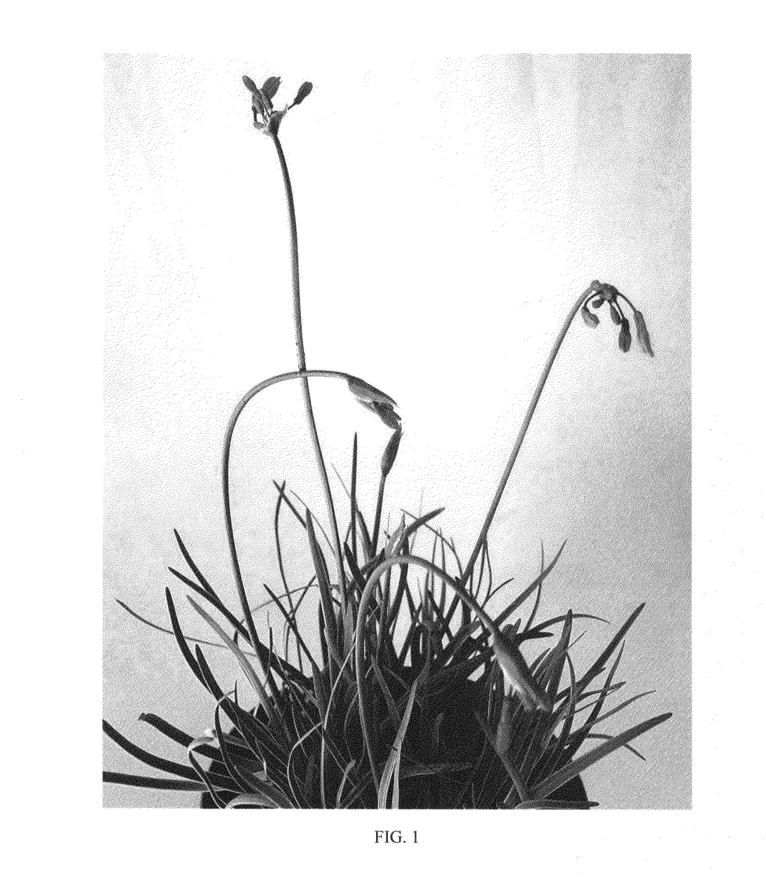

FIG. 2 shows a close-up of the foliage and inflorescence.

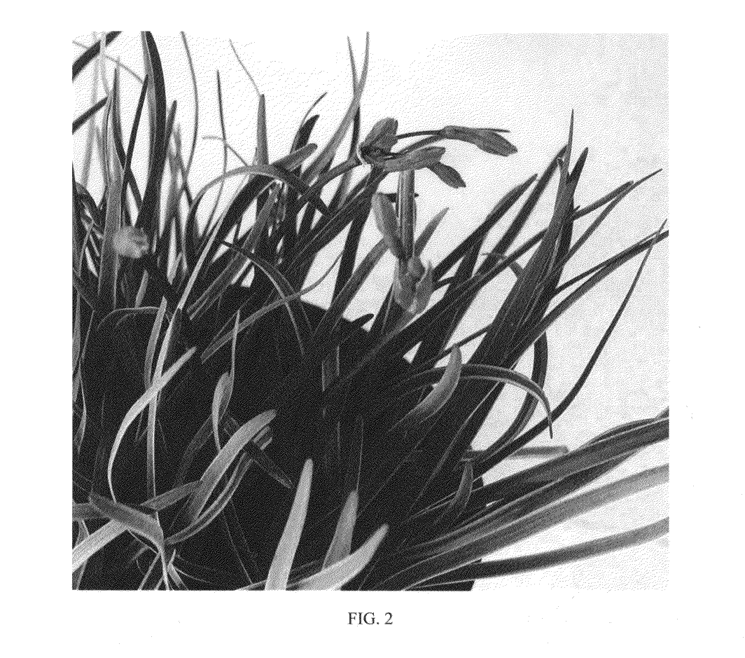

### DESCRIPTION OF THE NEW VARIETY

The following detailed descriptions set forth the distinctive characteristics of ‘Hinetul1’. The data which defines these characteristics were collected from asexual reproductions carried out in Rainbow, Calif. The plant history was taken in October 2012 on plants grown outdoors in Rainbow, Calif. Color references were taken under natural light and are to the R.H.S. Colour Chart of The Royal Horticultural Society of London (R.H.S.), third edition. Texture descriptions were taken using a dissecting microscope.
Classification:
   Family.—Amaryllidaceae.
   Botanical name.—Tulbaghia violacea.
   Denomination.—‘Hinetul1’.
Parentage:
   Mutation parent.—Tulbaghia violacea (unknown and unpatented).
Plant:
   Propagation type.—Divisions.
   Plant height (excluding inflorescence).—25.4 cm to 27.94 cm.
   Habit.—Upright growing with gently arching leaves growing from a central clump.
   Plant spread.—12.0 inches to 18.0 inches.
   Blooming period.—Year round in warmer climates, but natural blooming season is spring and summer.
   Hardiness.—Frost damage occurs when temperatures reach 20-25° F.
   Fragrance.—Flowers and stems have an onion or garlic odor.
   Plant disease/insect resistance/susceptibility.—None observed.
   Seed.—None observed.
   Market use.—For use in herb gardens, perennial gardens, mixed gardens, dry/rock gardens.
Foliage:
   Length.—17.78 cm to 30.48 cm.
   Width.—6.0 cm.
   Color.—

Upper surface: Repetitive and random stripes of RHS 137C, RHS 138D, and RHS 145D with RHS 74D on the margins Lower surface: Repetitive and random stripes of RHS 145D, RHS 138C, and RHS 137B with RHS 75A on the margins Texture (both upper and lower surfaces): Smooth Shape: Linear Apex: Rounded Base: Sheathed Margin: Entire

Inflorescence:

Height: 33.02 cm to 43.18 cm Umbel shape: Star shaped Umbel width: 2.54 cm to 5.08 cm Individual flower width: 1.59 cm Individual flower length: 1.4 cm Individual flower shape: Oblong Petals:

Upper surface: RHS 81D Lower surface: RHS 80C Scape (flowering stalk):

Length: 55.88 cm Diameter: 4.0 mm Color: RHS 90A

### Comparison with Commercial Variety

Ilinetull' differs from the commercial comparison Pertaylof (U.S. Plant Patent No. 9,019) in that Ilinetull' has purple inflorescences and purple and white variegated foliage, while Pertaylof has nearly white inflorescences and does not have variegated foliage.

I claim:

1. A new and dvariety of Tulbaghia plant as shown and described herein.
